(19) United States
(12) Patent Application Publication
(10) Pub. No.: US 2015/0087540 A1
(43) Pub. Date: Mar. 26, 2015

(54) Composition for Diagnosing Sepsis, and Method and Kit Therefor

(71) Applicants: M&D, INC., Gangwon-do (KR); YONSEI UNIVERSITY WONJU INDUSTRY-ACADEMIC COOPERATION FOUNDATION, Gangwon-do (KR)

(72) Inventors: Hye Young Lee, Gangwon-do (KR); Hye Young Wang, Jeollabuk-do (KR)

(21) Appl. No.: 14/241,869

(22) PCT Filed: Sep. 17, 2013
(86) PCT No.: PCT/KR2013/008431

§ 371 Date: Feb. 27, 2014

(30) Foreign Application Priority Data
Sep. 16, 2013 (KR) 10-2013-0111303

(51) Int. Cl. C12Q 1/68

(52) U.S. Cl. CPC C12Q 1/689; C12Q 2600/158; C12Q 2600/16
USPC 506/9; 506/16

(57) ABSTRACT

A composition for diagnosing sepsis, and a method and a kit therefor. The kit includes a primer composition containing specific individual primers for amplifying: a segment of a 16s rRNA gene; a segment of an ITS gene; a segment of a Nuc gene; a segment of a MecA gene; segments of a VanA and a VanB; a segment of an invA gene; a segment of an ipaH gene; and a segment of a Cps gene. The kit also includes a probe composition containing specific individual probes for detecting: gram-negative bacteria; gram-positive bacteria; a Nuc gene; a MecA gene for checking whether or not MRSA has antibiotic resistance; a VanA and a VanB, for checking whether or not VRE have antibiotic resistance; and a gene for identifying a fungus.

### TECHNICAL FIELD

The present invention relates to a composition for diagnosing sepsis, and a method and a kit therefor.

### BACKGROUND ART

Sepsis is a serious disease having a death rate of 23% to 46% depending on the time of detection of sepsis. Since sepsis significantly increases a financial burden, it is a big problem in health policy (Dellinger R P: Cardiovascular management of septic shock. Crit. Care Med 2003; 31: 946-55; Osborn T M, Tracy J K, Dunne J R, Pasquale \M, Napolitano L M: Crit. Care Med 2004; 32: 2234-40; Angus D C, Linde-Zwirble W T. Lidicker J, Clermont G, Carcillo J, Pinsky M R: Crit. Care Med 2001; 29: 1303-10).

Sepsis is the leading cause of death in non-cardiac intensive care units (ICUs), and in the United States, at least 750,000 cases of sepsis occurs each year, 50% of them proceed to septic shock cases, and half of the septic shock cases, i.e. 200,000 cases, die (Martin G S, Mannino D M, Eaton S, Moss M: N Engl J Med 2003; 348: 1546-54).

In recent decades, the incidence of septic shock is gradually on the rise, but its death rate has little changed or slightly decreased (Friedman G, Silva E, Vincent J L: Crit. Care Med 1998; 26: 2078-86).

Sepsis has complicated and various clinical signs since infection of a pathogenic microbe affects various functional systems of the body, such as the immune system, the coagulation system, the neurohormone system, etc. of a host, which results in a response of the whole body related thereto. Therefore, both a response degree of the host and a characteristic of the causative organism of the infection have significant effects on prognosis of sepsis.

Since the mid-1980s, gram-positive bacteria are mostly regarded as the cause of sepsis as compared with conventional gram-negative bacteria, and since 1990s, the incidence of sepsis caused by fungi has also been on the rise (Martin O S, Mannino D M, Eaton 5, Moss M: TN Engl J Med 2003; 348: 1546-54). It is deemed that such a change in strain causing sepsis is caused by an increase in elderly patients with associated diseases, improved and active medical and surgical treatment as compared with the past, an increase in diseases caused by human immunodeficiency virus, and expression of resistant bacteria having a resistance to conventional antibiotics.

Recently, a lot of yeast like fungi known as being non-pathogenic has emerged as critical opportunistic pathogens of immunodepressed patients. Opportunistic fungal infections often occur as complications in various medical and surgical inpatients such as a malignant tumor patient, an acquired immunodeficiency syndrome (AIDS) patient, a critical surgery patient, a severe burn patient, a hone-marrow or organ transplant patient, a patient undergoing intravascular catheter placement, a patient administered with antibiotics for a long time, and a patient undergoing chemotherapy (Kiehn T E, Edwards F F, Armstrong D. Am J Clin Pathol. 1980; 73:518-21; Komshian S V, Uwaydah A K, Sobel J D, Crane L R. Rev Infect Dis. 1989; 11:379-90). In addition to Candidal albican known as a common causative organism, Candida tropicalis and Candida parapsilosis among Candida spp. Have been on the rise in recent years (Goldani L Z and Mario P S. J. Infect. 2003; 46:155-60; Fraser V J, Jones M, Dunkel J, Storfer S, Medoff G, Dunagan W C. Clin Infect Dis. 1992; 15: 414-21), and it has been increasingly reported that as a result of preventive administration of fluconazole to hone-marrow transplant patients or the like, there occur opportunistic fungal infections caused by Candida krusei and Candida glabrata having a resistant to the fluconazole (Baran J Jr, Muckatira B, Khatib R. Scand J Infect Dis. 2001; 33:137-9; Diekema D J, Messer S A, Brueggemann A B, Coffman S L, Doern G V, Herwaldt L A, et al. J clin Microbiol. 2002; 40:1298-302; Collin B, Clancy C J, Nguyen M H. Drug Resist Updat. 1999; 2: 9-14).

For the past 70 years since 1940 when penicillin was first introduced to the clinical medicine, many antibiotics have been developed and have been used clinically, thereby having made a critical contribution toward saving numerous patients' lives from infections. However, along with use of antibiotics, antibiotic resistances of bacteria have been rapidly expressed and more rapidly progressed than development of antibiotics. Therefore, in only 70 years, curative effects of most of the antibiotics have been decreased all over the world. Considering the incidence and clinical importance of bacterial infections, antibiotic resistance can be said as a crisis in world's health care systems.

Major bacteria problematic regarding antibiotic resistance are Enterococcus faecium, Staphylococcus aureus, Klebsiella pneumoniae, Acinetobacter baumannii, Pseudomonas aeruginosa, Enterobacter spp. (Boucher H W, Talbot G H, Bradley J S, Edwards J E, Gilbert D, Rice L B, Scheld M, Spellberg B, Bartlett J. Badbugs, Clin Infect Dis 2009; 48: 1-12).

Particularly, as gram-positive bacteria, for example, Staphylococcus aureus has a representative resistance to methicillin (including community-acquired methicillin-resistant Staphylococcus aureus (MRSA)) and vancomycin, Enterococcus faecium has a representative resistance to vancomycin, and Streptococcus pneumoniae as a major community-acquired bacterium has a representative resistance to macrolide and multiple resistance.

MRSA has become the most critical nosocomial microbe in hospitals in the world. In particular, it is reported that in Korea, Japan, Taiwan, Hong Kong, Singapore, and Sri Lanka and in some hospitals in the United States, 50% or more of Staphylococcus aureus separated from pathogens are MRSA (Grundmann H, Aires-de-Sousa M, Boyce J, Tiemersma E. Lancet 2006; 368; 874-85).

The incidence of MRSA in the domestic hospitals was 83.7% as a result of the prospective research conducted in 15 domestic hospitals in 1996 (Kim J M, Park E S, Jeong J S, et al. Am J Infect control 2000; 28:454-8). According to the multi-institutional research from 1997 to 2006, the incidence of MRSA was reported as 64 to 72%, which confirmed that the MRSA is the most common causative organism of nosocomial infections in the domestic hospitals (Chong Y, Lee K, Park Y J, Jeon D S, et al. Yonsei Med J 1998; 39: 569-77).

Enterococcus was known as a major pathogen of endocarditis. However, as a use of third generation cephalosporin antibiotics has been increased since the mid-1970s, Enterococcus has become regarded as a major causative organism of nosocomial infections. Enterococcus has an inherent resistance to most of the antibiotics and can easily acquire antibiotic resistance through transfer of plasmid and transposon. Vancomycin-resistant Enterococci (VRE) were first reported in 1988 in Britain and France (Uttley A H, Collins C H, Naidoo J, George R C. Vancomycin-resistant Enterococci. Jancet 1988; 1: 57-8; Keclercq R, Duval J, Courvalin P. N Engl J Med 1988; 319: 157-61).

Since then, VRE showed a tendency to increase in the United States, and it was revealed that a transfer of VanA gene by means of transposon is a major mechanism thereof (Frieden T R, munsiff S S, Low D E et al. Lancet 1993; 342: 76-9).

In Korea, VRE infection was first reported in 1992. In Korea, the percentage of VRE among separated E. faecium was 4% in 1997 and has been gradually increased to 29% in 2009. According to a result of the nationwide multi-institution research, the percentage of E. faecium causing nosocomial infections from 2009 to 2010 was 38.9% (Park J W, Kim Y R, Shin W S, Kang M W, et al. Korean J Infect Dis 1992; 24: 133-7; Lee K, Kim M N, Kim J S et al, Yonsei Med J 2011; 52:793-802).

A blood culture is a very important method for testing bacteremia and a test method required for diagnosing a disease and determining a guideline for treatment and a prognosis (Aronson M I) and Bor D H. Blood culture. Ann Intern Med 1987; 106: 246-53).

Sepsis can be accompanied with various infectious diseases and can be caused by various bacterial species. Therefore, in order to identify a causative organism thereof, the blood culture is often used. By analyzing a result of the blood culture, studying development of changes thereof, and understanding a separated bacterial species and an antibiogram, important information for treatment of patients has been offered. Since a bacteremia patient is in a very serious condition, a fast result of the blood culture is very important in saving a life of the patient. However, the blood culture typically takes 5 days or more. If a blood culture-positive signal comes out, a subculture is carried out and then Gram staining, identification of a bacterial species, and an antimicrobial susceptibility test are carried out, which takes 10 days or more.

### PATENT LITERATURE

Patent Literature 1: Korean Patent Laid-open Publication No. 10-2005-0016987
Patent Literature 2: Korean Patent Laid-open Publication No. 10-2008-0006617

### DISCLOSURE

### Technical Problem

In order to solve the conventional problems, an object of the present invention is to provide an information offering method for diagnosing sepsis.

Another object of the present invention is to provide a primer for diagnosing sepsis.

Still another object of the present invention is to provide a probe for diagnosing sepsis.

Still another object of the present invention is to provide a kit for diagnosing sepsis.

### Technical Solution

In order to achieve the above objects, an exemplary embodiment of the present invention provides an information offering method for diagnosing sepsis comprising: (a) separating a DNA from a clinical specimen; (b) performing polymerase chain reaction (PCR) amplification of a 16s rRNA gene, an ITS (internal transcribed sequence) gene, a Nuc (heat-stable DNA nuclease) gene, a Cps gene (S. pneumoniae encoding biosyntheiss of capsular polysaccharide), a MecA gene (gene encoding methicillin resistance in saphylococci), an invA (invasion A) gene for detecting salmonella, an ipaH (invasion plasmid antigen) for detecting Shigella, and genetic fragments of a Van (Vancomycin resistance protein) A and a Van (Vancomycin resistance protein) B from the DNA by using respective primers; and (c) forming a PCR-reverse blot hybrid with a solid support upon which an oligomer probe for detecting the 16s rRNA gene for distinguishing gram-positive bacteria and gram-negative bacteria, an oligomer probe for detecting the ITS (internal transcribed sequence) gene for identifying a fungus, an oligomer probe for detecting the MecA gene for checking whether or not MRSA has antibiotic resistance, a probe for detecting the Nuc gene specific to S. aureus, a probe for detecting the Cps gene specific to S. pneumoniae, and a probe for detecting the invA gene for detecting salmonella, a probe for detecting the ipaH for detecting Shigella, and an oligomer probe for detecting the VanA and the VanB for checking whether or not VRE have antibiotic resistance are attached, and an amplified product obtained from the step (b).

In an exemplary embodiment of the present invention, preferably, a primer for amplifying a segment of the 16s rRNA gene is a primer having sequence numbers of 1 to 4, a primer for amplifying a segment of the ITS (internal transcribed sequence) gene is a primer having sequence numbers of 5 to 6, a primer for amplifying a segment of the Nuc (heat-stable DNA nuclease) gene is a primer having sequence numbers of 7 to 8, a primer for amplifying a segment of the MecA gene is a primer having sequence numbers of 9 to 10, primers for amplifying segments of the VanA and the VanB are primers having sequence numbers of 11 to 12 and sequence numbers of 13 to 14, respectively, a primer for amplifying a segment of the invA gene is a primer having sequence numbers of 15 to 16, a primer for amplifying a segment of the ipaH gene is a primer having sequence numbers of 17 to 18, and a primer for amplifying a segment of the Cps gene is a primer having sequence numbers of 19 to 20, but the present invention is not limited thereto.

In another exemplary embodiment of the present invention, preferably, a probe for detecting the gram-negative bacteria is a probe having sequence numbers of 22 to 30, a probe for detecting the gram-positive bacteria is a probe having sequence numbers of 31 to 37, a probe for detecting the Nuc (heat-stable DNA nuclease) gene is a probe having a sequence number of 38, a probe for detecting the MecA gene for checking whether or not MRSA has antibiotic resistance is a probe having a sequence number of 39, probes for detecting the VanA and the VanB for checking whether or not VRE have antibiotic resistance are probes having a sequence number of 40 and a sequence number of 41, respectively, and a probe for detecting the gene for identifying a fungus is a probe having sequence numbers of 42 to 47, but the present invention is not limited thereto.

Further, an exemplary embodiment of the present invention provides an information offering method for detecting gram-positive bacteria and gram-negative bacteria comprising: separating a DNA from a clinical specimen; and performing real-time PCR amplification using a primer pair having sequence numbers of 48 to 49 and a probe having sequence numbers of 50 to 53.

Furthermore, an exemplary embodiment of the present invention provides an information offering method for detecting a fungus comprising: separating a DNA from a clinical specimen; and performing real-time PCR amplification using a primer pair having sequence numbers of 54 to 55 and a probe having a sequence number of 56.

Moreover, an exemplary embodiment of the present invention provides a primer composition comprising: a primer having sequence numbers of 1 to 4; a primer having sequence numbers of 5 to 6 for amplifying a segment of an ITS (internal transcribed sequence) gene; a primer having sequence numbers of 7 to 8 for amplifying a segment of a Nuc (heat-stable DNA nuclease) gene; a primer having sequence numbers of 9 to 10 for amplifying a segment of a MecA gene; primers having sequence numbers of 11 to 12 and sequence numbers of 13 to 14 for amplifying segments of a VanA and a VanB, respectively; a primer having sequence numbers of 15 to 16 for amplifying a segment of an invA gene; a primer having sequence numbers of 17 to 18 for amplifying a segment of an ipaH gene; and a primer having sequence numbers of 19 to 20 for amplifying a segment of a Cps gene.

Besides, an exemplary embodiment of the present invention provides a probe composition comprising: a probe having sequence numbers of 22 to 30 for detecting gram-negative bacteria; a probe having sequence numbers of 31 to 37 for detecting gram-positive bacteria; a probe having a sequence number of 38 for detecting a Nuc (heat-stable DNA nuclease) gene; a probe having a sequence number of 39 for detecting a MecA gene for checking whether or not MRSA has antibiotic resistance; probes having a sequence number of 40 and a sequence number of 41 for detecting a VanA and a VanB, respectively, for checking whether or not VRE have antibiotic resistance; and a probe having sequence numbers of 42 to 47 for detecting a gene for identifying a fungus.

Further, an exemplary embodiment of the present invention provides a kit for diagnosing sepsis, the kit containing the primer composition and the oligomer probe composition of the present invention as active ingredients.

Furthermore, an exemplary embodiment of the present invention provides a composition for detecting gram-positive bacteria and gram-negative bacteria, the composition containing a primer pair having sequence numbers of 48 to 49 and a probe having sequence numbers of 50 to 53 as active ingredients.—

Moreover, an exemplary embodiment of the present invention provides a composition for detecting a fungus, the composition containing a primer pair having sequence numbers of 54 to 55 and a probe having a sequence number of 56 as active ingredients.

The primer of the present invention can be chemically synthesized by using a phosphoramidite solid support method or other well-known methods. This nucleic acid sequence can also be deformed by the known methods in the art. Non-limited examples of such deformation may include methylation, “capping”, substitution to analogues of at least one natural nucleotide, and deformation between nucleotides, for example, deformation into an uncharged connector (for example, methyl phosphonate, phosphotriester, phosphoramidate, carbamate, or the like) or a charged connector (for example, phosphorothioate, phosphorodithioate, or the like). The nucleic acid may contain at least one additional covalently bonded residue, for example, proteins (for example, nuclease, toxin, antibody, signal peptide, poly-L-lysine, or the like), an intercalator (for example, acridine, psoralene, or the like), a chelating agent (for example, a metal, a radioactive metal, iron, an oxidative metal, or the like), and an alkylating agent. The nucleic acid sequence of the present invention can also be deformed by using a marker capable of directly or indirectly providing a detectable signal. For example, the marker may include a radioactive isotope, a fluorescent molecule, biotin, and the like.

The term “real-time polymerase chain reaction (real-time PCR)” refers to a molecular biological polymerization method using a DNA as a template to amplify a target by using a target probe including a target primer and a marker and simultaneously quantitatively detect a signal generated from the marker of the target probe to the amplified target.

### Effect

According to the present invention, Real GP-GN/Real Can using a real-time PCR method which can rapidly distinguish gram positive bacteria, gram negative bacteria, and Candida species and can be substituted for the above-described blood culture and REBA (Reverse blot hybridization assay) Sepsis-ID as a rapid and accurate identification method capable of distinguishing gram positive bacteria and gram negative bacteria, identifying a fungus including Candida spp., and identifying whether or not MRSA and VRE have antibiotic resistance at the same time have been developed, and usefulness thereof have been confirmed.

### DESCRIPTION OF DRAWINGS

FIG. 1 shows a result of a PCR amplified product for REBA Sepsis-ID;

FIGS. 2 to 5 show results of specificity of REBA Sepsis-ID in a reference strain;

FIG. 6 shows a result of color development of REBA Sepsis-ID using a reference strain;

FIGS. 7 and 8 show result examples of REBA Sepsis-ID in a clinical blood culture strain;

FIG. 9A-D are provided to check sensitivity in gram-positive bacteria, gram-negative bacteria, and a fungus by using a real-time PCR; and

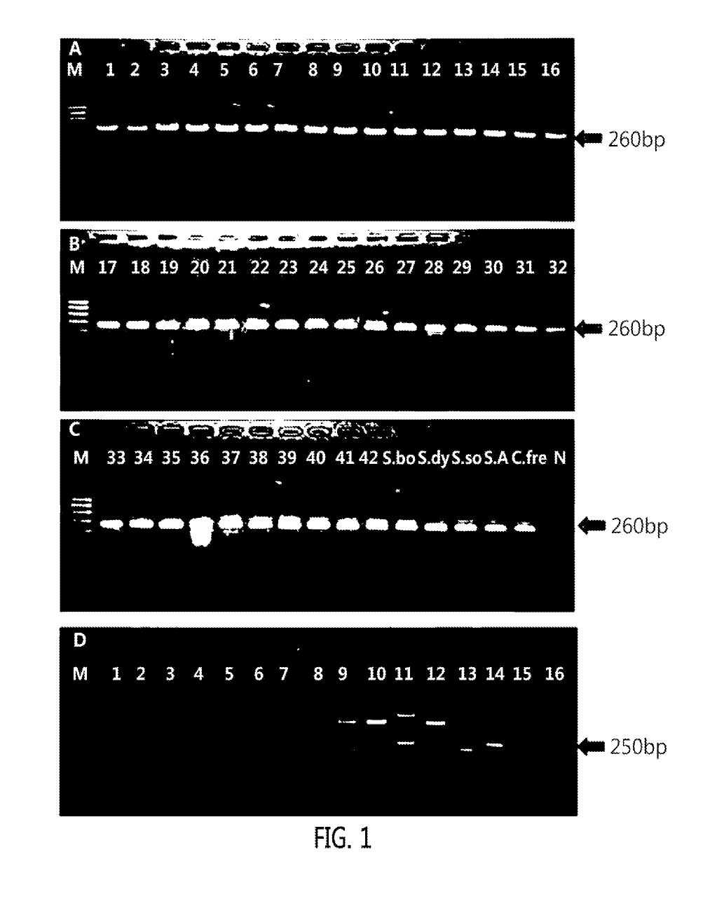

FIG. 1D shows Real GP-GN PCR negative and positive specimens in Lanes 1 to 21 as a result of REBA Sepsis-ID with respect to 21 negative and positive specimens from a blood culture.

### BEST MODE

Hereinafter, the present invention will be described in detail with reference to non-limited examples below. The following examples are provided for illustrating the present invention. Therefore, the scope of the present invention should not be construed as being limited by the following examples.

### Example 1

### Materials

Blood culture positive bacterial species from a blood culture specimen sent to the Department of the Laboratory Medicine of Wonju Severance Christian Hospital and a specimen having an antimicrobial susceptibility test result were used.

For a blood culture, 5 to 10 ml of blood from a suspected bacteremia case was inoculated into each of a pair of blood culture bottles including a BACTEC Standard 10 Aerobic/F bottle and a BACTEC PLUS Anaerobic/F bottle. All the blood culture bottles were received within 24 hours and cultured within 3 hours in two kinds of automated blood culture systems, a BACTEC 9240 system (BD, Franklin Lakes, N. J., USA) and a BacT/Alert 3D system (BioMerieux, Durham, N. C., *SA), at 37° C. for 5 days. When a blood culture-positive signal came out, a subculture was carried out and then Gram staining, identification of a bacterial species, and an antimicrobial susceptibility test were carried Out. The identification of a bacterial species was carried out by a biochemical method using a Vitek II system (BioMerieux, Marcy, l'Eltoile, France). As for a Candida species, after a germ tube test, if positive, the Candida species was identified as a Candida albicans, and if negative, the Candida species was identified by using a ATB ID 32C (BioMerieux S A, Marcy-l'Etoile, France).

### Example 2

### Extraction of Genomic DNA from Reference Strain and Clinical Isolate

A part of a culture medium cultured in a BACTEC 9240 system (Becton Dickinson Microbiology System, Sparks, Md, USA) was obtained. 100 μl of a DNA extraction solution was put into the obtained culture medium, vortexed for 1 minute, heated at 100° C. for 10 minutes, and centrifuged at room temperature at 13,000 rpm for 3 minutes so as to obtain a supernatant. A nucleic acid was separated by using this method. The separated nucleic acid was used as a template of a PCR for implementing Real GP-GN/Real Can and REBA Sepsis-ID.

### Example 3

### Gene Collection and Analysis in Real GP-GN/Real can and REBA Sepsis-ID

From Genbank of National center for biotechnology information (NCBI, http://www.ncbi.nlm.nih.gov)), as targeted PCR primers and oligonucleotide probes for accurate identification of a bacterial species of sepsis, a 16s rRNA gene for detecting gram-positive bacteria and gram-negative bacteria, an ITS (internal transcribed sequence) gene existing between 18S rRNA and 5.8S rRNA for detecting a fungus, a MecA gene for checking whether or not MRSA has antibiotic resistance, and VanA and VanB genes for checking whether or not VRE have antibiotic resistance were searched, and base sequences thereof were collected. After multi-alignment (http://multalin.toulouse.inra.fr/multalin) was carried out, two pairs of primers were designed at a base sequence site common to the targeted genes. At a reverse primer of them, biotin was attached to 5′-terminal for PCR-REBA. Then, oligonucleotide probes capable of detecting target bacterial species from pathogen-specific sites existing within the two pairs of primers were designed. At each of the oligonucleotide probes, an amine group was attached to 5′-terminal so as to form a peptide bond with a thin membrane. As for the designed primers and oligonucleotide probes, Bioneer (Daejeon, Korea) was requested to synthesize them.

### Example 4

### One-Tube Nested PCR

A PCR was carried out with the extracted genomic DNA of each strain as a template by using a commercialized Prime Taq Premix (2×) (Genet Bio, Nonsan, Korea). A composition of Prime Taq Premix (2×) included primer Taq polymerase I unit/10 μl, a 2× reaction buffer, 4 mM MgCl₂, an enzyme stabilizer, a sediment, a loading dye, pH 9.0, 0.5 mM of each dATP, dCTP, dGTP, and dTTP. Each composition for PCR included 10 μl of Prime Taq premix (2×), 1 μl of each of a pair of 10 pmole primers, 3 μl of ultrapure water, and 5 μl of the genomic DNA of each strain to be the total reaction amount of 20 μl. During the PCR, pre-denaturation at 95° C. for 5 minutes, primary amplification at 95° C. for 30 seconds, and a reaction at 60° C. for 30 seconds were carried out repeatedly 15 times; then secondary amplification at 95° C. for 30 seconds and a reaction at 54° C. for 30 seconds were carried out repeatedly 35 times; and then full extension at 72° C. for 10 minutes was carried out. After the PCR was completed, a PCR product was electrophoresed in a 2% TBE (Tris-borate-ethylenediaminetetraacetic acid disodium salt dehydrate) agarose gene (W/V ratio) at 290 bolt for 20 minutes and dyed in ethidium bromide for 10 minutes. Then, whether or not the PCR product was amplified was checked. Herein, a 16s rRNA capable of identifying GN-GP had a sequence of 280 bp, an ITS gene positioned between 18S and 5.8S rRNAs and capable of identifying a fungus had a sequence of 250 bp, a nuc gene specific to only S. aureus had a sequence of 136 bp, a Spn gene gene specific to only S. pneumoniae had a sequence of 120 hp, a MecA gene for checking methicillin resistance had a sequence of 140 bp, and VanA and VanB genes for checking vancomycin resistance had sequences of 170 hp and 100 hp, respectively.

### Example 5

### Implementation of REBA Sepsis-ID

REBA Sepsis-ID using the amplified PCR product was carried out in experimental conditions suggested by a manufacturer according to the following experiment method. The double stranded PCR product was mixed and reacted with an equivalent amount of a denaturation solution (0.2 N NaOH, 0.2 mM EDTA) at room temperature for 5 minutes to be single stranded. During the reaction, a thin REBA Sepsis-ID membrane (M&D, Korea) attached with a pathogen-specific oligonucleotide probe was diluted in 2× SSPE/0.1% SDS, put into a Membrane minitray (Bio-rad, USA), reacted at 55° C. for 30 minutes, washed two times with a WS (Washington solution) at 55° C. for 10 minutes, and reacted at room temperature for 30 minutes with an alkaline phosphatase-labeled Streptavidin conjugate (Roche, Mannheim, Germany) diluted at 1:2000 (v/v). After the reaction, the membrane was washed two times at room temperature for 1 minute with a SS (Staining solution, TBS pH 7.5), exposed for 10 minutes to NBT/BCIP (Nitro blue tetrazolium chloride and 5-Bromo-4-chloro-3-indolylphosphate, toluidine salt in 67% DMSO (v/v), Roche, Germany) as a staining solution reacting to the alkaline phosphatase, washed with DW, and then dried to determine whether or not there was detection.

### Example 6

### Implementation of Real GP-GN/Real Can

Real GP-GN/Real Can used the extracted genomic DNA of each strain as a template with addition of 10 μl of a 2× PCR premix (TOYOBO, Japan), 5 μl of a mixture of GP-GN primer/probe, and 5 μl of an extracted DNA specimen to be the total amount of 20 μl. PCR conditions included one-time pre-denaturation at 94° C. for 3 minutes, denaturation at 94° C. for 20 seconds, annealing at 60° C. for 40 seconds, extension, and a reaction using CFX 96 (Bio-Rad, USA) 40 times in total. Real GP-GN included a GP probe (HEX) for detecting gram-positive bacteria, a GN probe (FAM) for detecting gram-negative bacteria, and an internal control (IC) probe (Cy5). A measured threshold cycle (Ct) value was analyzed and determined as being positive if equal to or lower than 35 and determined as being negative if higher than 35. Further, Real Can used a Can probe (FAM) for detecting a Candida species, and in the same manner, a threshold cycle (Ct) value was analyzed and determined as being positive if equal to or lower than 35 and determined as being negative if higher than 35. Furthermore, when the IC probe and the GP probe were detected at the same time or when only the GP probe was detected, it was determined as gram-positive bacteria; when the IC probe and the GN probe were detected at the same time or when only the GN probe was detected, it was determined as gram-negative bacteria; and when the IP probe and the GN-GP probes were detected at the same time or when the GN-GP probes were detected at the same time, it was determined that gram-positive bacteria and gram-negative bacteria were mixed. However, when any signal from the probes including the IC probe was not detected, it was determined as failure of the test.

Results of Examples above were as shown below.

1. Result of PCR Amplification

PCR amplification products having a sequence of 280 hp could be observed from gram-positive bacteria such as E. facalis, E. solitarius, E. faecium, E. malodoratus, E. saccharolytivus, E. casseliflavus, S. pneumoniae, S. agalactiae, etc. (photograph A and B of FIG. 1) and gram-negative bacteria such as E. coli, K. pneumoniae, K. oxytoca, S. liquifaciens, P. alcalifaciens, P. vulgaris, P. mirabilis, S. typhi, etc. (photograph C of FIG. 1) using the 16s rRNA gene. Further, a PCR amplification product having a sequence of 250 hp could be observed from a Candida-related DNA such as C. albicans, C. tropicalis, C. glabrala, C. parapsilosis, etc. using the ITS gene between the 18S and 5.8S rRNAs (photograph D of FIG. 1).

2. Pathogen Specificity Test of GN-GP Bacteria Using Reference Strain

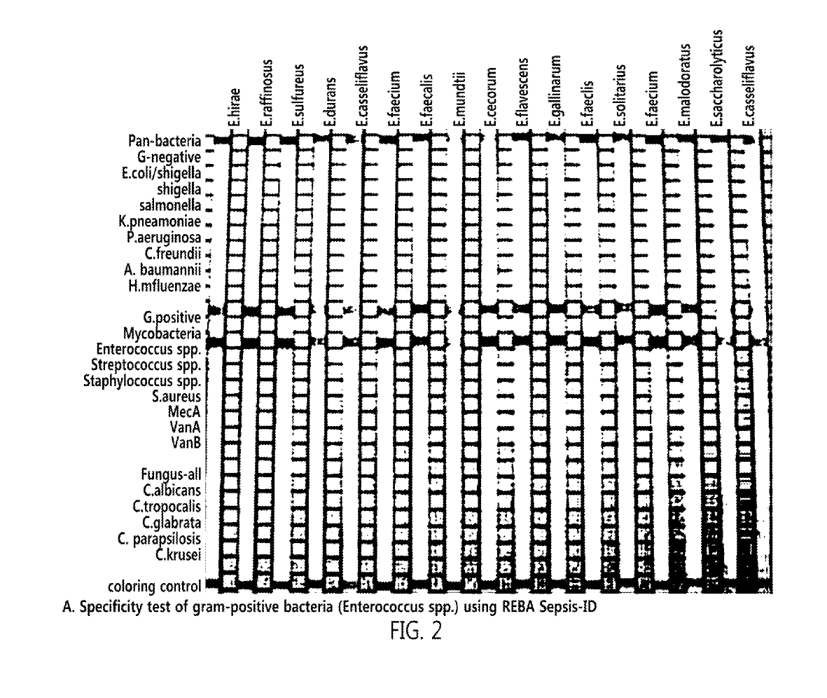

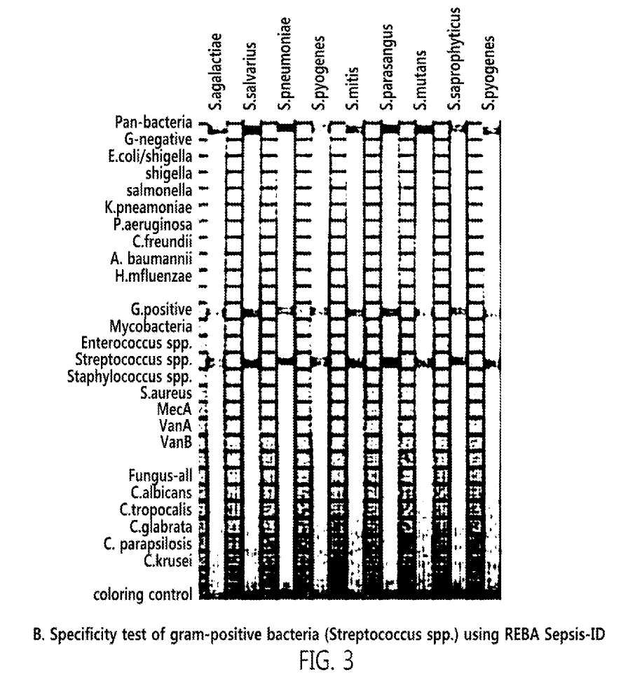

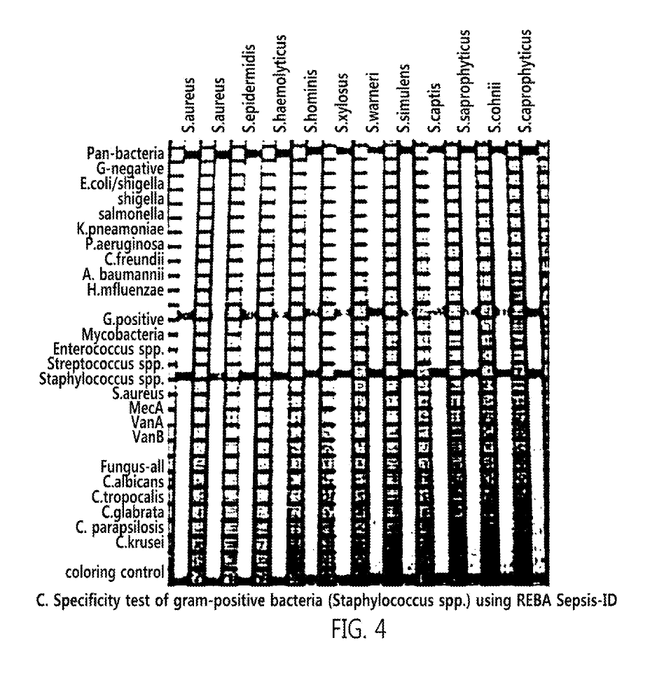

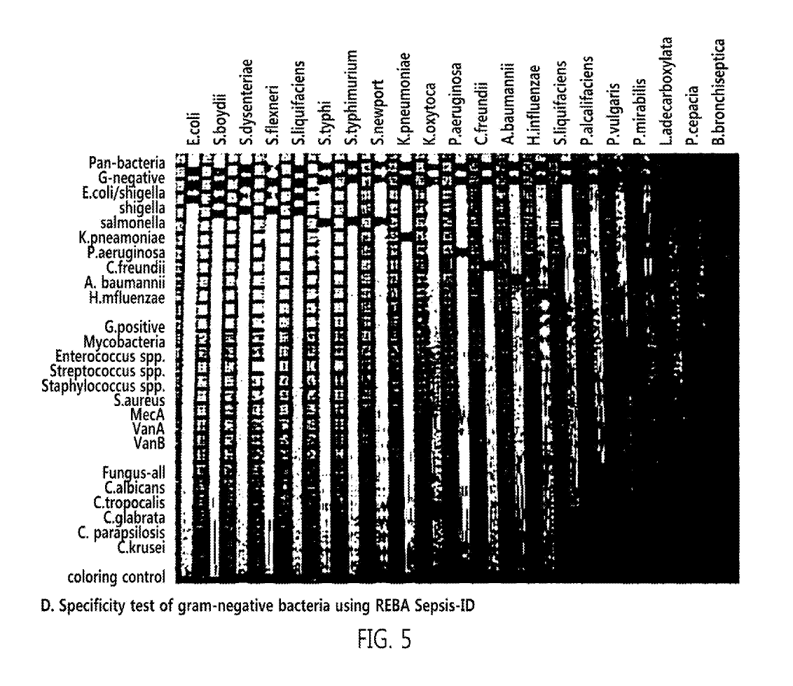

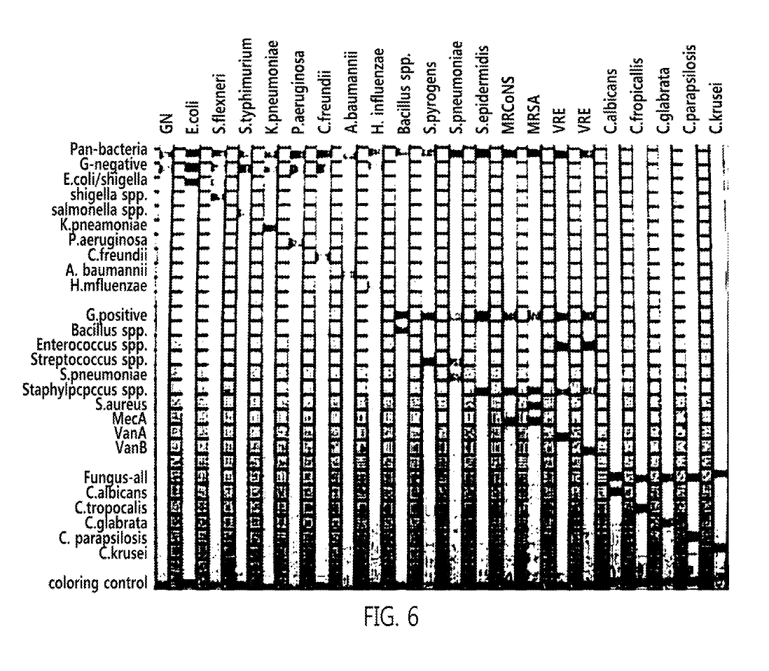

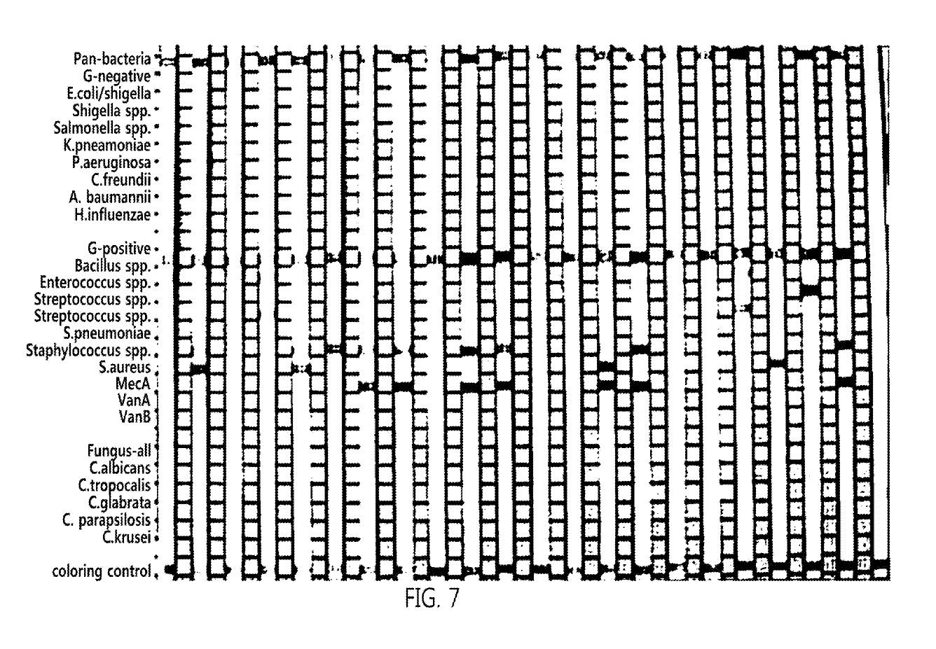

PCR-REBA was carried out to check specificity of the pathogen-specific oligonucleotide probe manufactured by using the PCR amplification product through a reference strain. As a result thereof, it could be confirmed that all of 38 gram-positive strains made positive reactions with the gram-positive probe, an Enterococcus spp. probe (FIG. 2), a Streptococcus probe (FIG. 3), and a Stapyhlococcus probe (FIG. 4), and all of 21 gram-negative strains made positive reactions the gram-negative probe and specific probes of respective bacterial species (FIG. 5). Further, a result of the staining using a membrane strip can be seen from FIG. 6.

3. Evaluation of Usefulness of PCR-REBA Using Clinical Solid Culture Strain

PCR-REBA was carried out to 118 solid culture strains in total including clinically separated 41 gram-positive strains, 64 gram-negative strains, and 13 fungi (Table 1). As a result thereof, it could be confirmed that all of the 118 strains were identified by respective probes for gram-positive bacteria, gram-negative bacteria, and fungi.

4. Evaluation of Usefulness of PCR-REBA Using Clinical Blood Culture Positive Strain

As a result of evaluating usefulness of REBA Sepsis-ID using 70 gram-positive strains, 32 gram-negative strains, and 13 fungi through clinical blood culture positive strains (FIG. 7), all of 13 Staphylococcus aureus specimens, 23 S. epidermidis specimens, 5 S. capitis specimens, 5 S. haemolyticus specimens, 5 S. hominis specimens, and a S. warneri specimen among the 70 gram-positive strains were identified by a Staphylococcus spp. probe; Streptococcus parasanguinis, each S. salivarius specimen was identified by a Streptococcus spp. probe, and it could be confirmed that all of 6 Enterococcus faecium specimens were detected by an Enterococcus spp. probe. An E. facalis specimen was detected by an Enterococcus spp. probe, and it could be confirmed that a Corynebacterium spp. specimen, 2 Micrococcus spp. specimens, a Propionibacterium acnes specimen, 5 Gram-positive rods specimens were detected by a GP probe. It was confirmed that among the 32 gram-negative strains, all of 14 Escherichia coli specimens, 7 Klebsiella pneumonia specimens, and each of Salmonella group, Haemophilus influenzae, Pseudomonas aeruginosa specimens were detected by the probes respectively corresponding thereto. It was confirmed that all of 2 K. oxytoca specimens and each of Acinetobacter lwoffii, Aeromonas spp., Citrobacter koseri, Neisseria sicca, Proteus mirabilis, Sphingomonas paucimobilis specimens were detected by a GN probe. Further, it was confirmed that among the 13 fungi specimens, all of 8 C. albicans specimens, 2 C. parapsilosis specimens, and a C. glabrata specimen were detected by the probes respectively corresponding thereto, and it was confirmed that a Cryptococcus neoformans and a Saccharomyces cerevisiae specimen were detected by Pan-fungus (Table 2).

5. Evaluation of Usefulness of MRSA and VRE Through Antibiotic Resistance Test Strain

As a result of checking whether or not the MecA gene was detected to check methicillin resistance by using the solid culture gram-positive strains, 9 of 12 S. aureus specimens were confirmed as MRSA, and 7 of 8 Staphylococcus spp. specimens were confirmed as MRCoNS, and, thus, detection of the MecA gene was confirmed (Table 1). Further, among the blood culture positive specimens, 10 of 13 S. aureus specimens were confirmed as MRSA, and 32 of 39 Staphylococcus spp. specimens were confirmed as MRCoNS, and, thus, existence of the MecA gene could be confirmed. In a test for checking vancomycin resistance regarding Enterococcus spp., it was confirmed that 7 of 16 solid culture strains had a resistance to the VanA gene and it was confirmed that 2 of 7 blood culture positive specimens had a resistance to the VanA gene (Table 2).

6. Check of Sensitivity of Real GP-GN/Real Can

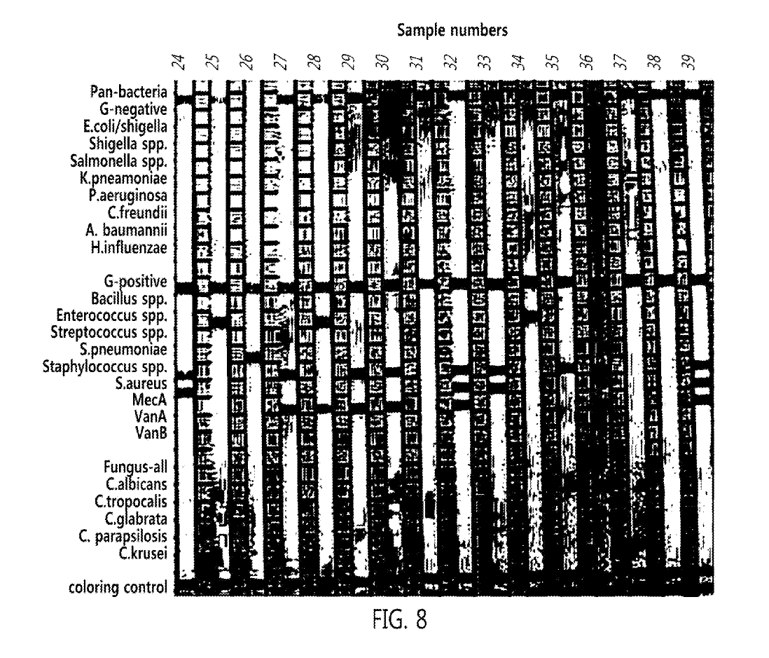

In order to rapidly check gram-positive bacteria, gram-negative bacteria, or fungi first during a blood culture, the Real-time PCR method was used. In order to do so, sensitivity in gram-positive bacteria, gram-negative bacteria, and fungi were checked by 10 times diluting DNAs of S. aureus as gram-positive bacteria, E. coli as gram-negative bacteria, and C. glabrata as fungi. As a result thereof, it could be found that the gram-positive bacteria and the gram-negative bacteria showed sensitivity in a range of 100 fg to 10 fg, and the fungi showed sensitivity of 1 pg (FIG. 8).

7. Result of Real GP-GN PCR Using Reference Strain

As a result of checking specificity of Real GP-GN by using 63 reference strain specimens, it could be confirmed that all of 39 gram-positive bacteria specimens were detected by a GP (HEX) probe, all of 24 gram-negative bacteria specimens were detected by a GN (FAM) probe, and all of 5 Candida strains were detected by a Can (FAM) probe (Tables 3 and 4).

8. Check of Usefulness of Real GP-GN PCR Using Solid Culture Bacteria

As a result of checking usefulness of Real GP-GN/Real Can by using 105 solid culture bacteria specimens, it could be confirmed that all of 41 gram-positive bacteria specimens including 12 S. aureus specimens were detected by a GP (HEX) probe with a Ct value of 17.35 to 30.46. Further, it could be confirmed that all of 64 gram-negative bacteria specimens including 16 E. coli specimens were detected by a GN (FAM) probe with a Ct value of 12.68 to 33.54, and it could be confirmed that all of 10 Candida specimens including 5 C. albicans specimens were detected by a Can (FAM) probe with a Ct value of 17.61 to 30.95 (Table 5).

9. Check of Usefulness of Real GP-GN PCR Using Blood Culture Positive Bacteria

As a result of checking usefulness of Real GP-GN by using 176 positive specimens from a blood culture bottle, all of 175 gram-positive bacteria specimens were detected by a GP (HEX) probe and a Ct value at that time was in a range of 12.43 to 34, and all of 70 gram-negative bacteria specimens were detected by a GN (FAM) probe and a Ct value at that time was in a range of 6.56 to 24.08. As a result of Real GP-GN PCR of a specimen obtained from the culture and including a mixture of S. agalactiae and C. koseri, Ct values of 14.61 and 10.61 were shown at GP and GN probes, respectively. Therefore, it was confirmed that even the mixed specimens could be detected accurately. Thus, it could be found that sensitivity of seroprevalence of the Real GP-GN PCR using the positive bacteria in the blood culture bottle was 99.6%. As a result of checking usefulness of Real Can by using 24 fungi specimens, 2 C. albicans specimens and each of C. parapsilosis and C. tropicalis specimens were detected by a Can (FAM) probe and a Ct value at that time was in a range of 22.81 to 31.98 (Tables 6 to 8).

10. Check of Usefulness of Real GP-GN PCR Using Blood Culture Negative Bacteria

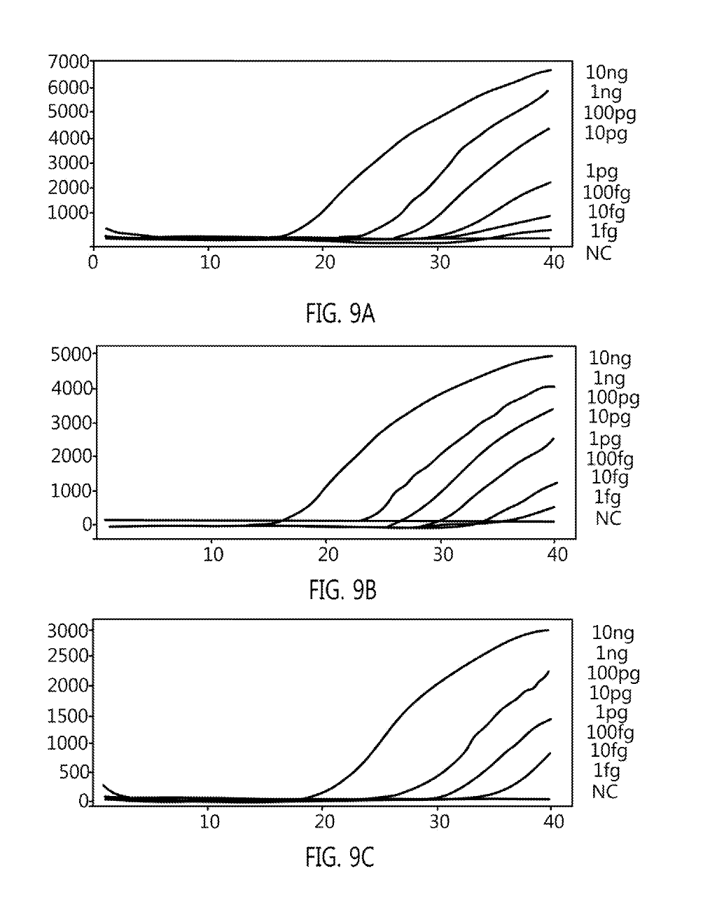

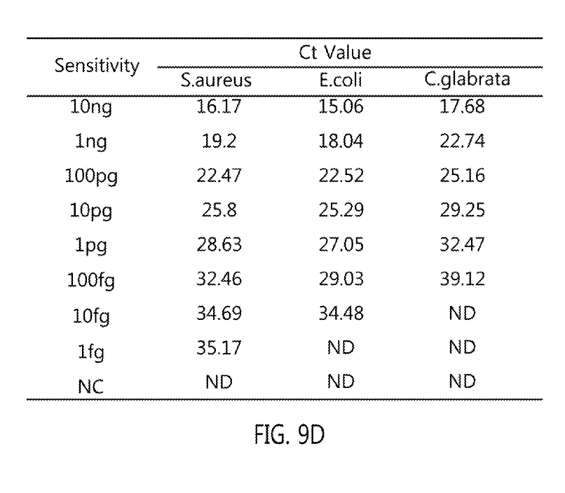

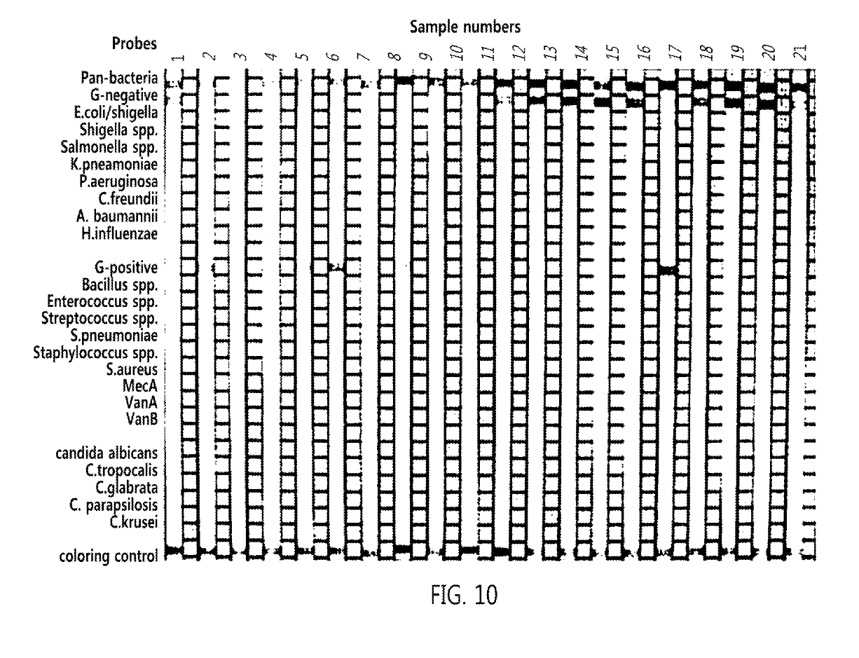

As a result of checking usefulness of Real GP-GN PCR by using 200 negative specimens from a blood culture bottle, 21 specimens in total including 7 gram-positive bacteria specimens by a GP (HEX) probe and 14 gram-negative bacteria specimens by a GN (FAM) probe were confirmed as being positive. Thus, it was found that specificity was 89.5% (Table 9). The positive specimens from the Real GP-GN PCR underwent PCR REBA and sequencing, and results thereof were compared (FIG. 9A-D). As a result of the PCR-REBA, 7 Real GP specimens were detected by a GP probe, and among 14 Real GN specimens, 12 specimens were detected by a GN probe, another specimen was detected by Pan-bacteria, and the other specimen was not detected. As a result of the sequencing, all of 7 gram-positive specimens were shown as uncultured bacteria. One of 14 gram-negative specimens was shown as an uncultured bacterium, and among the other 13 specimens, 2 Proteobacterium specimens and 2 Pseudomonas spp. specimens were detected and the other specimens were Janthinoba cterium sp., Polynucleobacter sp., Hyalangium sp., Duganella sp., Ochrobacterum sp., Burkholderiales bacterium, Methylophilus sp., Nitrosomonadaceae, and Enterobacter sp. (Table 10).

Although the conventional culture method has been used so far to check gram-positive bacteria and gram-negative bacteria, it is possible to rapidly and accurately check gram-positive bacteria and gram-negative bacteria through Real GP-GN in 1 hour to 1 hour and 30 minutes and also possible to check bacterial species of the gram-positive bacteria and gram-negative bacteria, methicilin resistance, and vancomycin resistance through REBA Sepsis-ID in further 1 hour to 1 hour to 30 minutes, i.e. 4 hours in total. Therefore, it can be substituted for the culture method in which a culture takes 3 to 5 days and then antibiotic resistance is checked and a result thereof can be obtained in further 5 to 7 days. Further, the Real GP-GN PCR method is a useful method which can be substituted for the culture method considering that even if a culture is carried out for 3 to 5 days, there may be a negative result and the culture negative bacteria as shown in the present experiment has a seroprevalence of about 10%.

TABLE 1

| | Number of isolates | | | |
|---|---|---|---|---|
| Conventional methods | | | REBA Sepsis-ID | Antibiotics resistance |
| Gram | Staphylococcus aureus | 12 | S. aureus | MRSA (9) |
| positives | S. epidermidis | 4 | Staphylococcus sp. | MRCoNS (4) |
| (41) | S. haemolyticus | 3 | Staphylococcus sp. | MRCoNS (3) |
| | S. capitis | 1 | Staphylococcus sp. | MRCoNS (1) |
| | Streptococcus agalactiae | 1 | Streptococcus sp. | |
| | S. mitis | 2 | Streptococcus sp. | |
| | S. parasanguis | 1 | Streptococcus sp. | |
| | S. salivorius | 1 | Streptococcus sp. | |
| | S. pyogenes | 1 | Streptococcus sp. | |
| | Enterococcus faecalis | 4 | Enterococcus sp. | |
| | E. faecium | 10 | Enterococcus sp. | VRE (7) |
| | E. mundtii | 1 | Enterococcus sp. | |
| | Corynebacterium spp. | 1 | Gram positives | |
| | Enterobacteriaceae | | | |
| Gram | Escheria coli | 16 | E. coli | |
| negatives | Enterobacter asburiae | 1 | Gram negatives | |
| (64) | E. cloacae | 1 | Gram negatives | |
| | Klebsiella pneumoniae | 14 | K. pneumoniae | |
| | Citrobacter freundii | 1 | C. freundii | |
| | Morganella morgannii | 1 | Gram negatives | |
| | Proteus mirabilis | 1 | Gram negatives | |
| | Serratio marcescens | 1 | Gram negatives | |
| | Providencia rettgeri | 1 | Gram negatives | |
| | Glucose non-fermenter | | | |
| | Acinetobacter baumannii | 11 | A. baumannii | |
| | Pseudomonas aeruginosa | 13 | P. aeruginosa | |
| | Others | | | |
| | Aeromonas spp. | 1 | Gram negatives | |
| | Haemophilus influenzae | 1 | H. influenzae | |
| | Moraxella catarrholis | 1 | Gram negatives | |
| Fungus | Candido albicans | 5 | C. albicans | |
| (13) | C. glabrata | 1 | C. glabrata | |
| | C. parapsilosis | 3 | C. parapsilosis | |
| | C. tropicalis | 2 | C. tropicalis | |
| | Saccharomyces cerevisiae | 2 | Fungi | |
| Total | | 118 | | 24 |

Table 1 shows a comparison between a culture method and a PCR-REBA method in 118 solid culture strains.

TABLE 2

| Conventional methods | | REBA Sepsis-ID | |
|---|---|---|---|
| Genus and species (n) | Antibiotic resistance | Genus and species (n) | Antibiotic resistance |

Gram-positive bacteria (70)

| | | | |
|---|---|---|---|
| Stahylococcus aureus (13) | MRSA (10) | S. aureus (13) | MRSA (10) |
| | MSSA (3) | | MSSA (3) |
| S. epidermidis (23) | MRCoNS (20) | Staphylococcus spp. (23) | MRCoNS (20) |
| | MSCoNS (3) | | MSCoNS (3) |
| S. capitis (5) | MRCoNS (3) | Staphylococcus spp. (5) | MRCoNS (3) |
| | MSCons (1) | | MSCons (1) |
| S. haemolyticus (5) | MRCoNS (5) | Staphylococcus spp. (5) | MRCoNS (5) |
| S. hominis (5) | MRCoNS (3) | Staphylococcus spp. (5) | MRCoNS (3) |
| | MSCoNS (2) | | MSCoNS (2) |
| S. warneri (1) | | Staphylococcus spp. (1) | |
| Streptococcus parasanguinis (1) | | Streptococcus spp. (1) | |
| S. salivarius (1) | | Streptococcus spp. (1) | |
| Enterococcus faecium (6) | VRE (2) | Enterococcus spp. (6) | |
| | VSE (4) | | |
| E. faecalis (1) | | Enterococcus spp. (1) | |
| Corynebacterium spp. (1) | | Gram positive (1) | |
| Micrococcus spp. (2) | | Gram positive (1) | |
| Propionibacterium acnes (1) | | Gram positive (1) | |
| Gram-positive rods (5) | | Gram positive (5) | |

Gram-negative bacteria (32)

| | |
|---|---|
| Escherichia coli (14) | E. coli (14) |
| Klebsiella pneumoniae (7) | K. pneumoniae (7) |
| K. oxytoca (2) | Gram negatives (2) |
| Salmonella group D (1) | Salmonella spp. (1) |
| Acinetobacter lwoffii (1) | Gram negatives (1) |
| Aeromonas spp. (1) | Gram negatives (1) |
| Citrobacter koseri (1) | Gram negatives (1) |
| Haemophilus influenzae (1) | H. influenzae (1) |
| Neisseria sicca (1) | Gram negatives (1) |
| Proteus mirabilis (1) | Gram negatives (1) |
| Pseudomonas aeruginosa (1) | P. aeruginosa (1) |
| Splingomonas paucimobilis (1) | Gram negatives (1) |

Fungi (13)

| | |
|---|---|
| Candida albicans (8) | C. albicans (8) |
| C. parapsilosis (2) | C. parapsilosis (2) |
| C. glabrata (1) | C. glabrata (1) |
| Cryptococcus neoformans (1) | Fungi (1) |
| Saccharomyces cerevisiae (1) | Fungi (1) |

Table shows a comparison between a culture method and a PCR-REBA method in 115 blood culture positive specimens.

TABLE 3

| | | | Real-time PCR TaqMan | | |
|---|---|---|---|---|---|
| | | | assay (C_{T} value) | | |
| Genus | Species | *ATCC no. | Real-GP ™ | Real-GN ™ | Real-CAN ™ |

Gram positive bacteria (39)

| | | | | | |
|---|---|---|---|---|---|
| Staphylococcus (3) | S. aureus | 29213 | 26.59 | ^{s}UD | UD |
| | S. aureus | 25923 | 28.42 | UD | UD |
| | S. xylosus | 29971 | 20.81 | UD | UD |
| Enterococcus (17) | E. hirae | 9790 | 26.31 | UD | UD |
| | E. raffinosus | 49427 | 28.09 | UD | UD |
| | E. sulfureus | 49903 | 28.40 | UD | UD |
| | E. durans | 19432 | 23.48 | UD | UD |
| | E. casseliflavus | 700327 | 25.98 | UD | UD |
| | E. faecium | 19434 | 26.72 | UD | UD |
| | E. faecalis | 29212 | 25.33 | UD | UD |
| | E. mundtii | 43186 | 29.90 | UD | UD |
| | E. cecorum | 43198 | 21.68 | UD | UD |
| | E. flavescens | 49997 | 22.21 | UD | UD |
| | E. gallinarum | 49573 | 23.88 | UD | UD |
| | E. faeclis | 51299 | 24.10 | UD | UD |
| | E. solitarius | 49428 | 30.44 | UD | UD |
| | E. faecium | 35667 | 24.66 | UD | UD |
| | E. malodoratus | 43197 | 27.02 | UD | UD |
| | E. saccharolyticus | 43076 | 23.06 | UD | UD |
| | E. casseliflavus | 25788 | 26.28 | UD | UD |
| Streptococcus (2) | S. puemoniae | 49619 | 21.11 | UD | UD |
| | S. agalactiae | 13813 | 26.41 | UD | UD |
| Micrococcus (1) | M. luteus | 49732 | 22.36 | UD | UD |
| Mycobacterium (16) | M. avium | 25291 | 23.06 | UD | UD |
| | M. chelonae | 35749 | 21.34 | UD | UD |
| | M. gastri | 15754 | 23.9 | UD | UD |
| | M. kcoisasii | 12478 | 22.17 | UD | UD |
| | M. nonchromogenicum | 19530 | 18.31 | UD | UD |
| | M. phlei | 11758 | 25.09 | UD | UD |
| | M. smegunatis | 19420 | 24.4 | UD | UD |
| | M. triviale | 23292 | 22.66 | UD | UD |
| | M. aurum | 23366 | 24.83 | UD | UD |
| | M. farcinogen | 35753 | 20 | UD | UD |
| | M. gilvum | 43909 | 19.4 | UD | UD |
| | M. ncoaurum | 25795 | 17.83 | UD | UD |
| | M. parafortninum | 19686 | 19.06 | UD | UD |
| | M. peregrinum | 14467 | 18.57 | UD | UD |
| | M. septicum | 700731 | 23.47 | UD | UD |
| | M. abscessus | 19977 | 21.73 | UD | UD |

Table 3 shows detection of specificity of Real GP-GN/Real Can PCR by using 39 reference strain specimens.

TABLE 4

| | | | Real-time PCR TaqMan | | |
|---|---|---|---|---|---|
| | | | assay (C_{T} value) | | |
| Genus | Species | *ATCC no. | Real-GP ™ | Real-GN ™ | Real-CAN ™ |

Gram-negative bacteria (24)

| | | | | | |
|---|---|---|---|---|---|
| Escherichia (2) | E. coli | 25922 | UD | 20.46 | UD |
| | E. coli | 35218 | UD | 17.90 | UD |
| Enterobacter (1) | E. aerogenes | 1304 | UD | 19.09 | UD |
| Citrobacter (1) | C. freundii | 6750 | UD | 14.19 | UD |
| Shigella (3) | S. boydii | ^{#}DMl.399 | UD | 25.59 | UD |
| | S. dysenteriae | DMl.400 | UD | 18.38 | UD |
| | S. flexneri | 9199 | UD | 21.82 | UD |
| Serratia (1) | S. liquifaciens | 27952 | UD | 24.96 | UD |
| Salmonella (5) | S. typhi | 19430 | UD | 16.54 | UD |
| | S. enteriridis | 13076 | UD | 20.15 | UD |
| | S. paratyphi | 11511 | UD | 19.61 | UD |
| | S. typhimuriun | 13311 | UD | 16.19 | UD |
| | S. newport | 6962 | UD | 17.22 | UD |
| Klebsiella (2) | K. pneumaniae | 13883 | UD | 20.60 | UD |
| | K. oxytoca | 700324 | UD | 21.87 | UD |
| Proteus (3) | P. alcalifaciens | 51902 | UD | 18.38 | UD |
| | P. vulgaris | 49132 | UD | 17.13 | UD |
| | P. unirabilis | 49132 | UD | 16.34 | UD |
| Pseudomonas (2) | P. cepacia | 25608 | UD | 19.66 | UD |
| | P. aeruginosa | 27853 | UD | 16.57 | UD |
| Acinetobacter (1) | A. baumannii | 17978 | UD | 19.69 | UD |
| Haemophilus (1) | H. influenzae | 49247 | UD | 18.61 | UD |
| Leclercia (1) | L. adecarboxylata | 23216 | UD | 15.70 | UD |
| Bordetella (1) | H. branchiseptica | 10580 | UD | 19.44 | UD |

Fungi

| | | | | | |
|---|---|---|---|---|---|
| Candida (5) | C. albicans | 36802 | UD | UD | 26.42 |
| | C. tropicalis | 14506 | UD | UD | 25.98 |
| | C. glabrata | 38326 | UD | UD | 17.09 |
| | C. parapsilosis | 7330 | UD | UD | 24.27 |
| | C. krusei | 20298 | UD | UD | 19.67 |

*ATCC: American type culture collection.
^{#}DML: Diagnostic Microbiology Laboratory. Biomedical laboratory science. Yonsci University

Table 4 shows detection of specificity of Real GP-GN/Real Can PCR by using 29 reference strain specimens.

TABLE 5

| | | Real-time PCR TaqMan | | |
|---|---|---|---|---|
| | | assay (C_{T} value) | | |
| Culture identification | No. of samples | GP/GN or Fungi | Ranged C_{T} Value | Mean C_{T} Value |
| Staphylococcus aureus | 12 | GP | 22.44-26.65 | 24.47 |
| Staphylococcus spp. (CoNS) | 8 | GP | 19.46-28.71 | 21.5 |
| Streptococcus spp. | 5 | GP | 17.35-30.46 | 24.25 |
| Enterococcus faecalis | 4 | GP | 25.2-27.3 | 26.37 |
| E. faecium | 10 | GP | 21.3-31.6 | 26.58 |
| E. mundtii | 1 | GP | 27.85 | 27.85 |
| Corynebacterium spp. | 1 | GP | 24.51 | 24.51 |
| Escherichia coli | 16 | GN | 12.68-30.65 | 23.26 |
| Klebsiella pneumoniae | 13 | GN | 15.48-26.08 | 20.73 |
| Pseudomonas aeruginosa | 13 | GN | 15.23-19.96 | 18.11 |
| Acinetobacter baumannii | 11 | GN | 18.09-24.65 | 21.04 |
| Enterobacter asburiae | 1 | GN | 15.79 | 15.79 |
| E. coloacae | 1 | GN | 15.41 | 15.41 |
| E. asburiae | 1 | GN | 15.79 | 15.79 |
| Moraxella catarrhalis | 1 | GN | 33.54 | 33.54 |
| Serratia marcescens | 1 | GN | 21.64 | 21.64 |
| Providencia rettgeri | 1 | GN | 24.3 | 24.3 |
| Morganella morganii | 1 | GN | 20.6 | 20.6 |
| Proteus mirabilis | 1 | GN | 24.88 | 24.88 |
| Aeromonas spp. | 1 | GN | 25.97 | 25.97 |
| Citrobacter fruendii | 2 | GN | 17.11-18.01 | 17.56 |
| Candida albicans | 5 | Can | 17.61-29.56 | 23.9 |
| C. parapsilosis | 3 | Can | 24.73-30.95 | 27.58 |
| C. tropicalis | 1 | Can | 26.62 | 26.62 |
| C. glabrata | 1 | Can | 17.68 | 17.68 |
| total | 115 | | | |

Table 5 shows detection of specificity of Real GP-GN/Rea. Can PCR by using 115 solid culture bacteria specimens.

TABLE 6

| | Real-time PCR | | | | | | |
|---|---|---|---|---|---|---|---|
| | (No. of samples) | | Sensitivity | Specificity | GP | GN | CAN |
| Blood culture result | Positive | Negative | (%) | (%) | (Ranged C_{τ}) | (Ranged C_{τ}) | (Ranged C_{τ}) |
| Blood Culture Positive (276) | 275 | 1 | 99.6% | — | | | |
| Gram positive bacteria (176) | 175 | 1 | 99.4% | — | | | |
| Staphylococccus epidermidis (47) | 47 | 0 | | | 13.08-33.99 | UD | UD |
| S. aureus (24) | 24 | 0 | | | 13.24-34.19 | UD | UD |
| S. hominis (17) | 16 | 1 | | | 13.69-30.00 | UD | UD |
| S. capitis (14) | 14 | 0 | | | 13.60-25.10 | UD | UD |
| S. haemolyticus (8) | 8 | 0 | | | 14.68-33.3 | UD | UD |
| S. warneri (1) | 1 | 0 | | | 18.50 | UD | UD |
| S. saprophyticus (1) | 1 | 0 | | | 16.57 | UD | UD |
| S. xylosus (1) | 1 | 0 | | | 21.23 | UD | UD |
| S. chleiferi (1) | 1 | 0 | | | 20.44 | UD | UD |
| Streptococcus salivarius (5) | 5 | 0 | | | 13.55-23.58 | UD | UD |
| S. mitis (4) | 4 | 0 | | | 11.52-23.12 | UD | UD |
| S. pneumoniae (4) | 4 | 0 | | | 16.37-17.77 | UD | UD |
| S. agalactiae (2) | 2 | 0 | | | | UD | UD |
| S. pyogenes (1) | 1 | 0 | | | | UD | UD |
| S. dysgalactiae (1) | 1 | 0 | | | 15.81 | UD | UD |
| S. parasangus (1) | 1 | 0 | | | 12.96 | UD | UD |
| Streptococcus spp. (2) | 2 | 0 | | | | UD | UD |
| Enterococcus faecium (8) | 8 | 0 | | | 26.43-27.54 | UD | UD |
| K. faecalis (1) | 1 | 0 | | | 14.50 | UD | UD |
| Micrococcus spp. (5) | 5 | 0 | | | 20.96-31.33 | UD | UD |
| Propionibacterium acnes (3) | 3 | 0 | | | 23.77-26.86 | UD | UD |
| Peptostreptococcus | 1 | 0 | | | 26.72 | UD | UD |
| asaccharolyticus (1) | | | | | | | |
| Peptostreptococcus micros (1) | 1 | 0 | | | 26 | UD | UD |
| Corynebacterium spp. (6) | 6 | 0 | | | 26.47 | UD | UD |
| Gram positive rods (16) | 16 | 0 | | | 12.43-34.39 | UD | UD |

Table 6 shows a comparison between a result of detection by Real GP-GN PCR from a blood culture and a result of a culture in a BACTEC 9240.

TABLE 7

| | Real-time PCR | | | | | | |
|---|---|---|---|---|---|---|---|
| | (No. of samples) | | Sensitivity | Specificity | GP | GN | CAN |
| Blood culture result | Positive | Negative | (%) | (%) | (Ranged C_{τ}) | (Ranged C_{τ}) | (Ranged C_{τ}) |
| Gram negative bacteria (70) | 70 | 0 | 100% | | | | |
| Escherichia coli (35) | 35 | 0 | | | UD | 9.86-21.89 | UD |
| Klebsiella pneumoniae (13) | 13 | 0 | | | UD | 12.84-13.2 | UD |
| Acintobacter baumannii (5) | 5 | 0 | | | UD | 10.53-11.89 | UD |
| A. lwoffii (1) | 1 | 0 | | | UD | 12.22-15.11 | UD |
| Enterobacter spp. (2) | 2 | 0 | | | UD | 6.56 | UD |
| Pseudomonas aeruginosa (3) | 3 | 0 | | | UD | 12.08 | UD |
| Salmonella group D (1) | 1 | 0 | | | UD | 13.62-21.48 | UD |
| Proteus mirabilis (1) | 1 | 0 | | | UD | 14.38 | UD |
| Aeromonas spp. (2) | 2 | 0 | | | UD | 20.1 | UD |
| Morganella morganii (1) | 1 | 0 | | | UD | 10.39 | UD |
| Haemophillus influenzae (1) | 1 | 0 | | | UD | 20.68 | UD |
| Chryseobacterium indologenes (1) | 1 | 0 | | | UD | 14.4 | UD |
| Sphingomonas paucimobilis (1) | 1 | 0 | | | UD | 24.08 | UD |
| Serratia marcescens (1) | 1 | 0 | | | UD | | UD |
| Citrobacter freundii (1) | 1 | 0 | | | UD | | UD |

Table 7 shows a comparison between a result of detection by Real GP-GN PCR from a blood culture and a result of a culture in a BACTEC 9240.

TABLE 8

| | Real-time PCR | | | | | | |
|---|---|---|---|---|---|---|---|
| | (No. of samples) | | Sensitivity | Specificity | GP | GN | CAN |
| Blood culture result | Positive | Negative | (%) | (%) | (Ranged C_{τ}) | (Ranged C_{τ}) | (Ranged C_{τ}) |
| Fungus** (24) | 24 | 0 | 100% | | | | |
| Candida albicans (2) | 2 | 0 | | | UD | UD | 26.98-31.52 |
| C. parapsilosis (1) | 1 | 0 | | | UD | UD | 31.98 |
| C. tropicalis (1) | 1 | 0 | | | UD | UD | 22.81 |
| *Multiple Infection (6) | 6 | 0 | 100% | | | | |
| Streptococcus agalactiae, | 1 | — | | | 14.61 | 10.61 | UD |
| Citrobacter koseri (1) | | | | | | | |
| Enterococcus faecium, | 1 | — | | | 17.18 | UD | 29.51 |
| Candida albicans (1) | | | | | | | |
| Enterococcus faeclis, | 1 | — | | | UD | 12.14 | UD |
| Proteus mirabilis (1) | | | | | | | |
| Escherichia coli, | 1 | — | | | UD | 13.25 | UD |
| Enterococcus gallinarym (1) | | | | | | | |
| Klebsiella pneumonia, | 1 | — | | | UD | 13.31 | UD |
| Enterococcus casseliflavus (1) | | | | | | | |
| Klebsiella pneumonia, | 1 | — | | | UD | 14.42 | UD |
| Enterobacter cloacae (1) | | | | | | | |
| Blood Culture Negative (200) | 21 | 179 | | 89.5 | | | |
| Gram positive bacteria | 7 | 0 | | | 17.54-27.43 | — | — |
| Gram negative bacteria | 14 | 0 | | | — | 20.48-31.26 | — |
| Fungi | 0 | 0 | | | — | — | — |

*GP-GN mixed bacteria
**The specificity test for real GP-GN PCR

Table 8 shows a comparison between a result of detection by Real GP-GN PCR from a blood culture and a result of a culture in a BACTEC 9240.

TABLE 9

| | Real GP-GN PCR | | | |
|---|---|---|---|---|
| Blood culture | Positive | Negative | Sensitivity | Specificity |
| Gram positive bacteria | 126 | | 100% | |
| (126) | | | | |
| Gram negative bacteria (37) | 37 | | 100% | |
| Total (184) | 184 | | 100% | |
| Culture Negative (200) | 21 | 179 | | 89.50% |
| Gram positive bacteria | 12 | | | |
| Gram negative bacteria* | 9 | | | |

Table 9 shows sensitivity and specificity of Real GP-GN PCR in a blood culture bottle.

TABLE 10

| Case | Real GP-GN | | | REBA |
|---|---|---|---|---|
| no. | PCR | C_{T} Value | Sequencing results | results |
| 1 | GN | 28.25 | Janthinobacterium sp. | GN |
| 2 | GP | 21.23 | Unculturued bacterium | GP |
| 3 | GN | 30.31 | Polynucleobacter sp. | ND |
| 4 | GP | 27.43 | Unculturued bacterium | GP |
| 5 | GP | 23.28 | Unculturued bacterium | GP |
| 6 | GP | 19.92 | Unculturued bacterium | GP |
| 7 | GP | 17.54 | Unculturued bacterium | GP |
| 8 | GP | 17.85 | Unculturued bacterium | GP |
| 9 | GN | 17.79 | Unculturued bacterium | Pan-bac |
| 10 | GN | 29.05 | proteobacterium | GN |
| 11 | GN | 27.01 | proteobacterium | GN |
| 12 | GN | 31.26 | Hyalangnum sp. | GN |
| 13 | GN | 20.48 | Duganella sp. | GN |
| 14 | GN | 30.24 | Ochrobacterium sp. | GN |
| 15 | GN | 22.79 | Pseudomonas sp. | GN |
| 16 | GP | 27.15 | Unculturued bacterium | GP |
| 17 | GN | 25.33 | Burkholderiales bacterium | GN |
| 18 | GN | 28.31 | Methylophilus sp. | GN |
| 19 | GN | 22.59 | Nitrosomonadaccae | GN |
| 20 | GN | 27.15 | Pseudomonas sp. | GN |
| 21 | GN | 17.79 | Enterobacter sp. | GN |

Table 10 shows a comparison between PCR-REBA and sequencing with respect to 21 Real GP-GN PCR positive and culture negative bacteria specimens.

TABLE 11

| No. | Gene | Sequence | Size | Modification | |
|---|---|---|---|---|---|
| 1 | 16s | 16S-F* | T AAY ACA TGC AAG TCG ARC G | | Biotin |
| | rRNA | | | | |
| 2 | | 16S-R5H-A5 | TGG CAC GDA GTT RGC CGK KGC TT | 470 bp | Biotin |
| 3 | | 407F | T AAY ACA TGC AAG TCG ARC G | | |
| 4 | | 280R* | TGT GGC YGR TCR YCC TCT CAG | 170 bp | Biotin |
| 5 | ITS | MF3 | AACGCANMTTGCRCYCHHTG | | |
| 6 | | CR3* | CAGCGGGTADYCCYACCTGA | 230 bp | Biotin |
| 7 | Nuc | nuc-F | AGCGATTGATGGTGATACGGT | | |
| 8 | | nuc-R* | ATGCACTTGCTTCAGGACCA | 135 bp | Biotin |
| 9 | MecA | MecA-F | GGTGTTGGTGAAGATATACCAAGTG | | |
| 10 | | MecA-R* | GAAAGGATCTGTACTGGGTTAATCAT | 145 bp | Biotin |
| 11 | vanA | vanA325-F | TCAATAGCGCGGACGAATTG | | |
| 12 | | R* | GCGGGAACGGTTATAACTGCGTTT | 150 bp | Biotin |
| 13 | vanB | vanBF-3 | TACCTACCCTGTCTTTGTGAAGCC | | |
| 14 | | R* | GCTGCTTCTATCGCAGCGTTTAGT | 100 bp | Biotin |
| 15 | invA | sal-F | TCTGGCAGTACCTTCCTCAGCC | | |
| 16 | | sal-R* | TCGACAGACGTAAGGAGGACAAGA | 120 bp | Biotin |
| 17 | ipaH | Shi-F | AGTTGCAGTCTCCTAGGTAAAGGG | | |
| 18 | | Shi-R* | ACTGCAAACTCTTCCATCTCTGCC | 100 bp | Biotin |
| 19 | spn- | 297F | GACCAATCGTTTAAATGCGACTTCT | | |
| | cpsA | | | | |
| 20 | | 416R* | GTCCCAGTCGGTGCTGTCACACT | 130 bp | Biotin |

Table 11 shows a primer for REBA Sepsis-1a

TABLE 12

| No. | Name | Sequence | Species |
|---|---|---|---|
| 21 | Pan-bac | AGYGGCGG ACGGGTGAGTAA | |
| 22 | GN350-5 | AACKGCGATCCCTAGCTGGTC | Gram negative |
| 23 | Eco-5 | GGAAGGGAGTAAAGTTAATACCTTTGCTCA | E.coli/Shigella |
| 24 | shi | AGTTCAGTAAGATGGTTGTGCGCA | Shigella |
| 25 | sal | CGGAAGCCTCCGCTAATTTGAT | Salmonella |
| 26 | kpn-11 | AAAAAAA GGTTAATAACCTCATCGATTGAC | K.pneumoniae |
| 27 | Pae | ATACGTCCTGAGGGAGAAAGTG | P.aeruginosa |
| 28 | Cfre | CGCAGAGGAGCTTGCTCCTTG | C.freundii |
| 29 | Aba-3 | AGCTTGCTACCGGACCTAGCG | A.baumannii |
| 30 | Hinf | CGTATTATCGGAAGATGAAAGTGC | H.influenzae |
| 31 | GP9 | CVACGATRCRTAGCCGAC | Gram positive |
| 32 | GP11 | AAAAAACGATRCRTAGCCGAC | Gram positive |
| 33 | Baci | AAACCGTTCRAATAGGGCG | Bacillus spp. |
| 34 | Ent-3 | GGATAACACTTGGAAACAGGTGC | Enterococcus spp. |
| 35 | Strep-4 | GCGTAGGTAACCTGCCTBRTAGCG | Streptococcus spp. |
| 36 | spn | TAGCAGATAGTGAGATCGAAAATGTTAC | S. pneumoniae |
| 37 | staphyl-9 | CAWAYGTGTAAGTAACTRTGCACRTCT | Staphylococcus spp. |
| 38 | nuc | TTGGTTGATACACCTGAAACAAAG | S. aureus |
| 39 | MecA | AGCTGATTCAGGTTACGGACAAGGT | MecA |
| 40 | VanA | TCGTATTCATCAGGAAGTCGAGCC | VanA |
| 41 | VanB | TCGTCCTTTGGCGTAACCAA | VanB |
| 42 | alb | AA TAGTGGTAAGGCGGGATC | C. albicans |
| 43 | | GTAGTGGTAAGGCGGGATCG | |
| 44 | tro | ACG TGGAAACTTATTTT AAGCGA | C. tropicalis |
| 45 | gla | AGCGCAAGCTTCTCTATTAATCTG | C. glabrata |
| 46 | para | AGGCG GA GTATAAACTAATGGATAGGT | C. parapsilosis |
| 47 | kru | AGCGGAGCGGACGACGTGTA | C. krusei |

Table 12 shows a probe for REBA Sepsis-ID.

TABLE 13

| No. | Gene | | Sequence | Size | Modification |
|---|---|---|---|---|---|
| 48 | 16S rRNA | 300F | ATTAGCTAGTWGGTRRGGTAANGGC | 120 bp | |
| 49 | | 420R | ACTGCTGCCTCCCGTAGGAGT | | |
| 50 | | GP350S1 | C AAG GCA ACG ATR CRTAGCCGAC | | HEX-BHQ1 |
| 51 | | GP350M1 | AAGGCKWCGACGGGTAGCCGGC | | HEX-BHQ1 |
| 52 | | GN325-3 | TCACCTAGGCGACGATCYSTAGCKGGT | | FAM-BHQ1 |
| 53 | | Pan-bac | GCCACAYTGGRACTGAGACACGG | | Cy5-BHQ2 |

Table 13 shows a primer and a probe for Real GP-GN.

TABLE 14

| No. | Gene | | Sequence | Size | Modification |
|---|---|---|---|---|---|
| 54 | can18-1 | F | AGCTCGTAGTTGAACYTTGGGCYTG | 120 bp | |
| 55 | | R | TCAAAGTAAWMGTCCTGGTTCGCC | | |
| 56 | | P | CCGRGYCTTTCCTTCTGGSTARCC | | FAM-BHQ1 |

Table 14 shows a primer and a probe for Real Can.

1. An information offering method for diagnosing sepsis comprising:
(a) separating a DNA from a clinical specimen;
(b) performing polymerase chain reaction (PCR) amplification of a 16s rRNA gene, an ITS (internal transcribed sequence) gene, a Nuc (heat-stable DNA nuclease) gene, a Cps gene (S. pneumoniae encoding biosyntheiss of capsular polysaccharide), a MecA gene (gene encoding methicillin resistance in saphylococci), an invA (invasion A) gene for detecting salmonella, an ipaH (invasion plasmid antigen) for detecting Shigella, and genetic fragments of a Van (Vancomycin resistance protein) A and a Van (Vancomycin resistance protein) B from the DNA by using respective primers; and
(c) forming a PCR-reverse blot hybrid with a solid support upon which an oligomer probe for detecting the 16s rRNA gene for distinguishing gram-positive bacteria and gram-negative bacteria, an oligomer probe for detecting the ITS (internal transcribed sequence) gene for identifying a fungus, an oligomer probe for detecting the MecA gene for checking whether or not MRSA has antibiotic resistance, a probe for detecting the Nuc gene specific to S. aureus, a probe for detecting the Cps gene specific to S. pneumoniae, and a probe for detecting the invA gene for detecting salmonella, a probe for detecting the ipaH for detecting Shigella, and an oligomer probe for detecting the VanA and the VanB for checking whether or not VRE have antibiotic resistance are attached, and an amplified product obtained from the step (b).

2. The information offering method for diagnosing sepsis of claim 1, wherein a primer for amplifying a segment of the 16s rRNA gene is a primer having any of SEQ ID NOs.:1 to 4, a primer for amplifying a segment of the ITS (internal transcribed sequence) gene is a primer having any of SEQ ID NOs.:5 to 6, a primer for amplifying a segment of the Nuc (heat-stable DNA nuclease) gene is a primer having any of SEQ ID NOs.:7 to 8, a primer for amplifying a segment of the MecA gene is a primer having any of SEQ ID NOs.:9 to 10, primers for amplifying segments of the VanA and the VanB are primers having any of SEQ ID NOs.:11 to 12 and any of SEQ ID NOs.:13 to 14, respectively, a primer for amplifying a segment of the invA gene is a primer having any of SEQ ID NOs.:15 to 16, a primer for amplifying a segment of the ipaH gene is a primer having any of SEQ ID NOs.:17 to 18, and a primer for amplifying a segment of the Cps gene is a primer having any of SEQ ID NOs.:19 to 20.

3. The information offering method for diagnosing sepsis of claim 1, wherein a probe for detecting the gram-negative bacteria is a probe having any of SEQ ID NOs.:22 to 30, a probe for detecting the gram-positive bacteria is a probe having any of SEQ ID NOs.:31 to 37, a probe for detecting the Nuc (heat-stable DNA nuclease) gene is a probe having a any of SEQ ID NOs.:38, a probe for detecting the MecA gene for checking whether or not MRSA has antibiotic resistance is a probe having SEQ ID NO.:39, probes for detecting the VanA and the VanB for checking whether or not VRE have antibiotic resistance are probes having SEQ ID NO.:40 and of SEQ ID NO.:41, respectively, and a probe for detecting the gene for identifying a fungus is a probe having any of SEQ ID NOs.:42 to 47.

4-10. (canceled)

11. A kit for diagnosing sepsis, wherein the kit comprising a primer composition comprising:
a primer having any of SEQ ID NOs.:1 to 4 for amplifying a segment of a 16s rRNA gene; a primer having any of SEQ ID NOs.:5 to 6 for amplifying a segment of an ITS (internal transcribed sequence) gene; a primer having any of SEQ ID NOs.:7 to 8 for amplifying a segment of a Nuc (heat-stable DNA nuclease) gene; a primer having any of SEQ ID NOs.:9 to 10 for amplifying a segment of a MecA gene; primers having any of SEQ ID NOs.:11 to 12 any of SEQ ID NOs.:13 to 14 for amplifying segments of a VanA and a VanB, respectively; a primer having any of SEQ ID NOs.:15 to 16 for amplifying a segment of an invA gene; a primer having any of SEQ ID NOs.:17 to 18 for amplifying a segment of an ipaH gene; and a primer having any of SEQ ID NOs.:19 to 20 for amplifying a segment of a Cps gene; and
a probe composition comprising:
a probe having any of SEQ ID NOs.:22 to 30 for detecting gram-negative bacteria; a probe having any of SEQ ID NOs.:31 to 37 for detecting gram-positive bacteria; a probe having SEQ ID NO.:38 for detecting a Nuc (heat-stable DNA nuclease) gene; a probe having SEQ ID NO.:39 for detecting a MecA gene for checking whether or not MRSA has antibiotic resistance; probes having SEQ ID NOs.:40 and SEQ ID NO.:41 for detecting a VanA and a VanB, respectively, for checking whether or not VRE have antibiotic resistance; and a probe having any of SEQ ID NOs.:42 to 47 for detecting a gene for identifying a fungus.


SEQUENCE LISTING (56 sequences)

SEQ ID NO: 1 - DNA, 20 bp, Artificial Sequence
  primer
taayacatgcaagtcgarcg

SEQ ID NO: 2 - DNA, 23 bp, Artificial Sequence
  primer
tggcacgdagttrgccgkkgctt

SEQ ID NO: 3 - DNA, 20 bp, Artificial Sequence
  primer
taayacatgcaagtcgarcg

SEQ ID NO: 4 - DNA, 21 bp, Artificial Sequence
  primer
tgtggcygrtcrycctctcag

SEQ ID NO: 5 - DNA, 20 bp, Artificial Sequence
  primer
aacgcanmttgcrcychhtg

SEQ ID NO: 6 - DNA, 20 bp, Artificial Sequence
  primer
cagcgggtadyccyacctga

SEQ ID NO: 7 - DNA, 21 bp, Artificial Sequence
  primer
agcgattgatggtgatacggt

SEQ ID NO: 8 - DNA, 20 bp, Artificial Sequence
  primer
atgcacttgcttcaggacca

SEQ ID NO: 9 - DNA, 25 bp, Artificial Sequence
  primer
ggtgttggtgaagatataccaagtg

SEQ ID NO: 10 - DNA, 26 bp, Artificial Sequence
  primer
gaaaggatctgtactgggttaatcat

SEQ ID NO: 11 - DNA, 20 bp, Artificial Sequence
  primer
tcaatagcgcggacgaattg

SEQ ID NO: 12 - DNA, 24 bp, Artificial Sequence
  primer
gcgggaacggttataactgcgttt

SEQ ID NO: 13 - DNA, 24 bp, Artificial Sequence
  primer
tacctaccctgtctttgtgaagcc

SEQ ID NO: 14 - DNA, 24 bp, Artificial Sequence
  primer
gctgcttctatcgcagcgtttagt

SEQ ID NO: 15 - DNA, 22 bp, Artificial Sequence
  primer
tctggcagtaccttcctcagcc

SEQ ID NO: 16 - DNA, 24 bp, Artificial Sequence
  primer
tcgacagacgtaaggaggacaaga

SEQ ID NO: 17 - DNA, 24 bp, Artificial Sequence
  primer
agttgcagtctcctaggtaaaggg

SEQ ID NO: 18 - DNA, 24 bp, Artificial Sequence
  primer
actgcaaactcttccatctctgcc

SEQ ID NO: 19 - DNA, 25 bp, Artificial Sequence
  primer
gaccaatcgtttaaatgcgacttct

SEQ ID NO: 20 - DNA, 23 bp, Artificial Sequence
  primer
gtcccagtcggtgctgtcacact

SEQ ID NO: 21 - DNA, 20 bp, Artificial Sequence
  probe
agyggcggacgggtgagtaa

SEQ ID NO: 22 - DNA, 21 bp, Artificial Sequence
  probe
aackgcgatccctagctggtc

SEQ ID NO: 23 - DNA, 30 bp, Artificial Sequence
  probe
ggaagggagtaaagttaatacctttgctca

SEQ ID NO: 24 - DNA, 24 bp, Artificial Sequence
  probe
agttcagtaagatggttgtgcgca

SEQ ID NO: 25 - DNA, 22 bp, Artificial Sequence
  probe
cggaagcctccgctaatttgat

SEQ ID NO: 26 - DNA, 30 bp, Artificial Sequence
  probe
aaaaaaaggttaataacctcatcgattgac

SEQ ID NO: 27 - DNA, 22 bp, Artificial Sequence
  probe
atacgtcctgagggagaaagtg

SEQ ID NO: 28 - DNA, 21 bp, Artificial Sequence
  probe
cgcagaggagcttgctccttg

SEQ ID NO: 29 - DNA, 21 bp, Artificial Sequence
  probe
agcttgctaccggacctagcg

SEQ ID NO: 30 - DNA, 24 bp, Artificial Sequence
  probe
cgtattatcggaagatgaaagtgc

SEQ ID NO: 31 - DNA, 18 bp, Artificial Sequence
  probe
cvacgatrcrtagccgac

SEQ ID NO: 32 - DNA, 21 bp, Artificial Sequence
  probe
aaaaaacgatrcrtagccgac

SEQ ID NO: 33 - DNA, 19 bp, Artificial Sequence
  probe
aaaccgttcraatagggcg

SEQ ID NO: 34 - DNA, 23 bp, Artificial Sequence
  probe
ggataacacttggaaacaggtgc

SEQ ID NO: 35 - DNA, 24 bp, Artificial Sequence
  probe
gcgtaggtaacctgcctbrtagcg

SEQ ID NO: 36 - DNA, 28 bp, Artificial Sequence
  probe
tagcagatagtgagatcgaaaatgttac

SEQ ID NO: 37 - DNA, 27 bp, Artificial Sequence
  probe
cawaygtgtaagtaactrtgcacrtct

SEQ ID NO: 38 - DNA, 24 bp, Artificial Sequence
  probe
ttggttgatacacctgaaacaaag

SEQ ID NO: 39 - DNA, 25 bp, Artificial Sequence
  probe
agctgattcaggttacggacaaggt

SEQ ID NO: 40 - DNA, 24 bp, Artificial Sequence
  probe
tcgtattcatcaggaagtcgagcc

SEQ ID NO: 41 - DNA, 20 bp, Artificial Sequence
  probe
tcgtcctttggcgtaaccaa

SEQ ID NO: 42 - DNA, 20 bp, Artificial Sequence
  probe
aatagtggtaaggcgggatc

SEQ ID NO: 43 - DNA, 20 bp, Artificial Sequence
  probe
gtagtggtaaggcgggatcg

SEQ ID NO: 44 - DNA, 23 bp, Artificial Sequence
  probe
acgtggaaacttattttaagcga

SEQ ID NO: 45 - DNA, 24 bp, Artificial Sequence
  probe
agcgcaagcttctctattaatctg

SEQ ID NO: 46 - DNA, 27 bp, Artificial Sequence
  probe
aggcggagtataaactaatggataggt

SEQ ID NO: 47 - DNA, 20 bp, Artificial Sequence
  probe
agcggagcggacgacgtgta

SEQ ID NO: 48 - DNA, 25 bp, Artificial Sequence
  primer
attagctagtwggtrrggtaanggc

SEQ ID NO: 49 - DNA, 21 bp, Artificial Sequence
  primer
actgctgcctcccgtaggagt

SEQ ID NO: 50 - DNA, 23 bp, Artificial Sequence
  probe
caaggcaacgatrcrtagccgac

SEQ ID NO: 51 - DNA, 22 bp, Artificial Sequence
  probe
aaggckwcgacgggtagccggc

SEQ ID NO: 52 - DNA, 27 bp, Artificial Sequence
  probe
tcacctaggcgacgatcystagckggt

SEQ ID NO: 53 - DNA, 23 bp, Artificial Sequence
  probe
gccacaytggractgagacacgg

SEQ ID NO: 54 - DNA, 25 bp, Artificial Sequence
  primer
agctcgtagttgaacyttgggcytg

SEQ ID NO: 55 - DNA, 24 bp, Artificial Sequence
  primer
tcaaagtaawmgtcctggttcgcc

SEQ ID NO: 56 - DNA, 24 bp, Artificial Sequence
  probe
ccgrgyctttccttctggstarcc